(19) United States
(12) Patent Application Publication
(10) Pub. No.: US 2025/0381548 A1
(43) Pub. Date: Dec. 18, 2025

(54) DEVICE FOR CARRYING OUT A CHEMICAL REACTION IN A PLASMA AND METHOD USING THE DEVICE

(71) Applicant: FLD TECHNOLOGIES GMBH, Offenbach am Main (DE)

(72) Inventor: Marek FULDE, Offenbach (DE)

(21) Appl. No.: 18/877,472

(22) PCT Filed: Jun. 16, 2023
(86) PCT No.: PCT/EP2023/066241

§ 371 Date: Dec. 20, 2024

(30) Foreign Application Priority Data
Jun. 24, 2022 (EP) 22180957.7
Jul. 1, 2022 (EP) 22182584.7

(51) Int. Cl. B01J 19/12; B01J 4/00; B01J 6/00; B01J 19/00; C01B 3/24; C01B 3/34; C01B 32/05; C07C 2/80

(52) U.S. Cl. CPC B01J 19/12; B01J 4/001; B01J 6/008; B01J 19/0013; C01B 3/24; C01B 3/342; C01B 32/05; C07C 2/80; B01J 2204/002; B01J 2219/00087; B01J 2219/0871; B01J 2219/0875; B01J 2219/0883; B01J 2219/0898; B01J 2219/18; C01B 2203/0216; C01B 2203/0222; C01B 2203/0272; C01B 2203/1241

(57) ABSTRACT

The invention relates to a device (4) for carrying out a chemical rection in a plasma (223), wherein the device (4) comprises a source for generating electromagnetic waves (211), at least one first reactor (200), at least one connecting piece (230) and a second reactor (240). The invention also relates to a method for carrying out the chemical reaction using the device (4).

The invention relates to an apparatus for conducting a chemical reaction in a plasma, wherein the apparatus comprises a source for generation of electromagnetic waves and at least one reactor. The invention further relates to a method for conducting the chemical reaction using the apparatus.

The chemical reaction of gaseous reactants takes place in the plasma, involving a solid material which is formed or converted. The treatment or reaction of gases in a plasma can be divided into two categories. Firstly, thermal plasma which is formed on application of high-energy sources, such as a light arc, is used. The thermal plasma is characterized by temperatures of about 10 000° C. and is formed between two electrodes that are subject to wear. Thermal plasma is used, for example, for production of industrial carbon black.

Also known is nonthermal plasma, which is characterized in that only the electrons of the gas attain a very high energetic state, while ions or free radicals have a significantly lower energetic state. The nonthermal plasma has a lower temperature range from 1000° C. to 5000° C. Electromagnetic waves are frequently used for induction of a nonthermal plasma. Nonthermal plasma in particular is used for the treatment of gases, especially for chemical conversions.

M. Jasinski et al., in “Studies of atmospheric-pressure microwave plasmas used for gas processing”, Nukleonika 2012, 57 (2), pages 241 to 247, describe several methods for gas treatment. This distinguishes between three practically relevant types of atmospheric plasma sources.

Firstly, in the case of surface wave discharge, plasma is generated within a quartz tube. Secondly, in the case of the nozzle-based solution, plasma is formed in a microwave field, with the plasma gas flowing out of a nozzle. Thirdly, in the case of a tube-based solution, plasma gas flows out of a cylindrical tube. In these plasma-generating sources, a quartz tube that bounds the plasma is subjected to high temperatures and has to be cooled. This considerably reduces the effectiveness of the process. Moreover, solid particles such as soot that are involved in the chemical reaction are precipitated on the inner surface of the tube, absorb the electromagnetic waves and lead to overheating of the tube, which can lead to destruction thereof. A gas flow in vortex form in the same direction as the plasma flow is described for stabilization of the plasma and for cooling of the tube, but this cannot prevent deposition of the solid particles.

US 2015/0174550 describes a method of treatment of a reaction product that has formed following a plasma-based reaction. The product is formed in a nozzle- or tube-based plasma gas apparatus. Here too, there is deposition of solids, and hence there are unfavorable thermal conditions in plasma formation as a result of unfavorable flow conditions in the reactor. Two gas streams are contacted only after one gas stream has been converted to a plasma. Effective mixing of gas and plasma is difficult since there is a considerable difference in density and viscosity. This has an adverse effect on the chemical reaction.

WO 2012/147054 discloses a tube closed at both ends for cracking of methane. A smaller tube is disposed at one end of the reactor, out of which a gas from the interior of the reactor tube can flow. The reaction gas is fed to the reactor at the same end of the tube. The cracking of methane is conducted in a plasma which is induced by electromagnetic waves. The reactants are mixed before being fed into the reactor, such that separate adjustment of the temperature of the individual reaction participants is impossible. The reaction of the mixture of the reactants starts on passage through a microwave window, which leads to soot formation on the window and hence to overheating of the window.

One object of the invention is to provide an apparatus and a method, with avoidance of the disadvantages from the prior art. The deposition of solid particles on the reactor wall and especially on the window for passage of electromagnetic waves is to be reduced or prevented. In addition, the mixing of different gas streams within the reactor is to be enabled or improved, so as to form a homogeneous reaction mixture and a stable plasma.

### DISCLOSURE OF THE INVENTION

What is proposed is an apparatus for conducting a chemical reaction in a plasma. This apparatus comprises a source for generation of electromagnetic waves, at least one first reactor, at least one connecting piece and a second reactor, wherein the first reactor, the connecting piece and the second reactor are each designed as tubes, the connecting piece has a smaller diameter than the first reactor, and the source for generation of electromagnetic waves is disposed on the first reactor, wherein the first reactor has a first outer face and first end faces, and the second reactor has a second outer face and second end faces, and the second reactor has an inner tube disposed at least partly within the second reactor, so as to form an inner gas space and an outer gas space that are separated from one another at the second end face of the second reactor that is closer to the connecting piece, wherein the connecting piece fluidically interconnects the first reactor and the second reactor, and the connecting piece exits from the first reactor at one of the first end faces and opens into the outer gas space of the second reactor at the second outer face, especially in tangential direction, and wherein the first outer face of the first reactor has a first section having at least one inlet for supply of a first input gas, a second section having at least one inlet for supply of a second input gas, and a window which is transparent to the electromagnetic waves and has been produced in particular from quartz, alumina, boron nitride or polytetrafluoroethylene, wherein the inlet for supply of the first input gas and the inlet for supply of the second input gas are aligned tangentially to the first outer face of the first reactor and the first section and the second section are in an axially offset arrangement, wherein the second section lies closer than the first section to the first end face from which the connecting piece exits from the first reactor, and the window is disposed between the first section and the second section and the second reactor, at least at one of the second end faces, has an outlet for removal of a product stream and the second outer face of the second reactor has a third section having at least one inlet for supply of a third input gas, and the third section is especially disposed at one of the second end faces of the second reactor that is further away from the connecting piece.

Also proposed is a method of conducting the chemical reaction using the apparatus of the invention, wherein a solid material, especially carbon is involved and is especially formed or converted, and the chemical reaction is preferably selected from the group consisting of pyrolysis of hydrocarbons, especially of methane, production of acetylene, reforming of hydrocarbons, especially methane, pyrolysis of hydrogen sulfide, pyrolysis of ammonia and hydrogasification of carbon.

The first reactor and the second reactor form a two-stage reactor cascade. The first reactor and the second reactor, i.e. the two stages, are connected to one another by the connecting piece.

The inlet in the first section or in the second section of the first outer face results in formation of two vortexlike flows close to the first outer face, i.e. the inner wall of the tube, which move toward one another. The outer faces may each also be regarded as a shell which, in particular, has only a low thickness relative to the reactor diameter. In the vicinity of the end of the first reactor that is further away from the connecting piece, a mixing zone is formed, i.e. in the first section in which the first input gas and the second input gas are mixed vigorously and form an inner vortex that moves in the direction of the connecting piece.

Plasma is injected at the window disposed between the first section and the second section, for example in the middle in axial direction of the first reactor. A plasma means a mixture of electrons, ions and free radicals. The inner vortex flows through the plasma zone and undergoes intense energization, which leads to initiation of chemical reactions in the gas phase.

The tangential feed, optionally disposed on different sides of the window, of the first input gas and of the second input gas into the first reactor results in formation of two vortex flows that move toward one another, which especially meet outside the window and lead to mixing of the first input gas with the second input gas, forming an input gas mixture. The input gas mixture flows past the window of the first reactor and is converted by the incoming electromagnetic waves to a plasma, which then leaves the first reactor via the connecting piece and enters the second reactor. For this purpose, the plasma is directed from the interior of the first reactor into the connecting piece.

The vortex flows also result in stabilization of the plasma in the first reactor, and the tangential gas flow at the reactor wall, i.e. the first outer face, avoids deposition of solids on the outer face and/or on the window.

As a result of the inventive feeding, the first input gas and the second input gas are mixed with one another if the two gases are still in a gaseous state. Only thereafter are the mixed gases converted collectively to a plasma, such that differences in density and viscosity between the gaseous state and the plasma state are not a barrier to mixing. The source for generation of electromagnetic waves serves for plasma generation. The first input gas is preferably a reaction gas. The second input gas is preferably an additional gas which is used in particular to promote plasma formation.

The energy needed for the chemical reaction to proceed is supplied to the input gas mixture at the window, i.e. in a plasma zone, in the form of electromagnetic waves. The second input gas comprising inert gases, for example, may serve to enhance plasma formation, especially in a case in which the actual reaction gas reacts less sensitively to electromagnetic waves and a plasma is less easily formed therefrom. The inert gases may also be regarded as energy carriers since they attain a high energetic state in the plasma and can subsequently pass energy to the actual reaction gas.

The connecting piece is especially in a tangential arrangement in or at the second outer face of the second reactor, i.e. the second stage of the cascade, such that a vortexlike flow is generated in the second reactor as well. The plasma formed is preferably conducted tangentially into the second reactor. Here too, a tangential feed of the plasma into the outer gas space of the second reactor leads to formation of a vortex flow that extends through the second reactor, optionally in contact with the third input gas, especially with a third input gas stream, and then leaves the second reactor at one of the second end faces. The first end faces and the second end faces may also be referred to as ground faces.

Tangential alignment exists in particular with regard to the first outer face of the first reactor or the second outer face of the second reactor, which especially form the inner surface of the reactor wall.

The inlets for feeding in the first, second or third input gas may also be referred to as gas addition apparatuses, which may especially be designed in the form of tangentially aligned holes through the outer wall, i.e. the respective outer face. The tangential alignment is based in particular on the circumference of the first reactor or of the second reactor.

The inlets are especially each formed by holes, preferably straight holes, where a drilling direction and the first outer face or the second outer face preferably have an angle of less than 45°, further preferably less than 30° and even further preferably less than 20°, especially less than 10°. As a result, the first input gas, the second input gas or the third input gas initially flows essentially parallel to the first outer face of the first reactor or the second outer face of the second reactor, and in circumferential direction, such that a near-wall vortex flow is formed within the first reactor or within the second reactor.

The vortex flows form flow conditions in the reactors, which are similar to those of an ideal tubular reactor.

The first reactor, the second reactor and/or the connecting piece are preferably each designed as cylindrical tubes. The cross-sectional area of the first reactor, the second reactor and/or the connecting piece is preferably circular, elliptical or polygonal, such as rectangular. In particular, the cross-sectional area of the first reactor, the second reactor and/or the connecting piece is circular.

The connecting piece preferably has a diameter at least 30% smaller than a reactor diameter of the first reactor, based on the reactor diameter of the first reactor. The inner tube of the second reactor preferably has a diameter at least 30% smaller than a reactor diameter of the second reactor, based on the reactor diameter of the second reactor.

The outer gas space is especially bounded by an inner wall of the second reactor, i.e. the second outer face, an outer wall of the inner tube, and one of the two end faces of the second reactor. The inner gas space is especially bounded by an inner wall of the inner tube and optionally by a lateral face of the inner tube.

The at least one inlet for supply of the first input gas and the at least one inlet for supply of the second input gas are especially on opposite sides of the window, such that the first section and the second section are separated from one another by the window in axial direction of the first reactor.

The second section and hence the inlet of the second input gas is disposed on the same side of the window as the connecting piece. Thus, the second input gas in the form of a vortex first flows past the window in the direction of the first section with the inlet for supply of the first input gas and is mixed therewith, forming the input gas mixture. The input gas mixture flows past the window into the connecting piece and is converted to a plasma at the window, which is spatially stabilized by the vortex flow of the second input gas that surrounds the plasma. The first input gas and the second input gas preferably form vortex flows in the first reactor that move toward one another. In addition, the flow of the second input gas preferably moves toward the plasma. The overall flow direction of the second input gas, especially on entry into the reactor, is preferably the opposite of the overall flow direction of the plasma. The plasma leaves the first reactor through the connecting piece and is transferred into the second reactor.

In the second reactor, a further vortex flow is preferably formed, especially around the inner tube. According to the arrangement of the inner tube in the second reactor, the product gas formed from the chemical reaction leaves the second reactor in the vicinity of the connecting piece or at the second end face further away from the connecting piece. If the outlet of the product gas stream is disposed on the side of the connecting piece at the second reactor, a further vortex flow is formed in the inner gas space, i.e. within the inner tube, in the direction of the outlet. In this embodiment, the vortex flow in the inner gas space moves toward the vortex flow in the outer gas space.

The second reactor is especially closed at both second end faces, except for the outlet of the product stream. The inner tube is preferably conducted through one of the second end faces, especially through the end face closest to the connecting piece. At the opposite end is preferably disposed the at least one inlet of the third input gas. The third input gas is preferably also introduced tangentially into the second reactor.

The inner tube is preferably secured at the second end face of the second reactor closest to the connecting piece. The outlet from the second reactor may be disposed at this second end face or at the opposite second end face of the second reactor. In one embodiment, the inner tube has an open end and a closed end, where the open end is disposed at the outlet of the second reactor. In a second embodiment, the inner tube has two open ends, where one of the two open ends forms the outlet of the second reactor. One of the second end faces may be open and form the outlet.

The second reactor may have a conical end, where the outlet is preferably disposed at one conical end, and the conical end further preferably forms the end of the second reactor further away from the connecting piece. In particular, one of the second end faces may be in conical form. In this case, the inner tube preferably has an open end and a closed end, where the open end is preferably disposed at the conical end of the second reactor.

The second reactor may have at least one inlet for supply of a third input gas, which is preferably disposed at the second end face of the second reactor that is further away from the connecting piece. The product gas may be mixed with the third input gas. The third input gas especially has a lower temperature within a range from 273 K to 1000 K. The third input gas thus serves to cool the plasma or the product gas.

The diameter of the connecting piece is preferably chosen such that spreading of the electromagnetic waves in the connecting piece is no longer possible. In this way, the effect of the electromagnetic field on the first reactor is limited. The reaction initiated in the first reactor is preferably continued in the connecting piece.

The connecting piece serves in particular as a barrier for the electromagnetic waves, which thus do not spread into the connecting piece and the second reactor. The connecting piece preferably has a constant diameter. A frequency f of the electromagnetic field and the diameter D of the connecting piece preferably satisfy the condition D*f<175.7, where the diameter D is given in mm and the frequency f in GHz. The first reactor and/or the second reactor preferably each have a constant reactor diameter. The reactor diameter of the second reactor is preferably chosen depending on the reaction kinetics. The reactor diameter of the second reactor is preferably equal to or greater than, more preferably greater than, the diameter of the connecting piece. The greater reactor diameter of the second reactor serves to form the vortex flow, which can also be referred to as swirl flow, in the second reactor.

A biphasic flow is preferably conducted through the connecting piece. The flow rate in the connecting piece is preferably at least 20 m/s.

The first reactor and the connecting piece are preferably arranged parallel to one another. The second reactor and the inner tube are preferably arranged parallel to one another. Further preferably, the first reactor and the connecting piece are in a coaxial arrangement. Further preferably, the second reactor and the inner tube are in a coaxial arrangement.

The inner tube preferably extends from the second end of the second reactor closer to the connecting piece up to a length within a range from 50% to 70% of a total length of the second reactor.

The connecting piece, which can also be referred to as plasma conductor, preferably has a length within a range from 30 to 1000 mm. In addition, the length of the connecting piece is preferably twice to 50 times the reactor diameter of the first reactor, more preferably 5 times to 25 times, especially 5 times to 10 times. The length of the connecting piece is preferably adjusted such that the plasma is extinguished in the connecting piece or on entry into the second reactor. This means that the subsequent reactions take place in the second reactor.

The source for generation of electromagnetic waves preferably comprises a wave channel that leads to the window, such that the electromagnetic waves can penetrate into the first reactor. A distance between an end of the window facing the connecting piece and the first end face at which the connecting piece is disposed is preferably 20% to 50%, further preferably 20% to 40% and especially preferably 20% to 25% of a total length of the first reactor, especially in axial direction.

The window is preferably formed over the entire circumference of the first reactor. The window preferably occupies 5% to 30% of a total area of the first outer face of the first reactor.

What is meant in particular by a material transparent to the electromagnetic waves is that the material has a low dielectric constant, i.e. low relative permittivity. Preferred materials transparent to the electromagnetic waves are materials having a dielectric constant of less than 10, especially polytetrafluoroethylene (PTFE), boron nitride, quartz, silicon dioxide and/or aluminum oxide.

The connecting piece is preferably straight. The first reactor and the second reactor are preferably arranged at an angle to one another within a range from 60° to 120°, especially from 80° to 100°, based on the center axes of the first reactor or of the second reactor.

The connecting piece preferably extends into the first reactor via the at least one inlet of the second section up to no further than the window. The connecting piece thus preferably projects into the first reactor, especially proceeding from one of the first end faces. The first input gas preferably at first forms a vortex flow around the connecting piece. The connecting piece extends no further than as far as the window, in order to avoid masking of the incoming electromagnetic waves. The continuation of the connecting piece within the first reactor serves for accommodation and stabilization, i.e. the spatial constriction, of the plasma, such that it continues to exist at least as far as the inlet into the second reactor.

The first section and/or the second section preferably each have at least two inlets. The at least two inlets are further preferably arranged opposite one another, based on a center axis of the first reactor. The first input gas and/or the second input gas is preferably divided between the respective at least two inlets. Feeding in the first input gas or the second input gas at at least two positions on the circumference of the first reactor promotes the formation of vortex flows. The at least two inlets of the first section or of the second section are preferably disposed at the same axial height of the first reactor.

The apparatus may have at least two, especially four, six, eight or twelve or more, first reactors each having one connecting piece. Further preferably, the connecting pieces each open tangentially into the outer gas space of the second reactor. In particular, each first reactor has a source for generation of electromagnetic waves. A plasma is preferably generated in each of the first reactors, and these are combined in the second reactor and form a common product gas.

The connecting piece and/or the inner tube may be equipped with a vibration apparatus. The vibration apparatus, which is especially electrical, can set the connecting piece and/or the inner tube in continuous or phased oscillation. The vibration apparatus can remove deposited solid particles or additionally prevent deposition.

At least parts of the first outer face of the first reactor and/or the connecting piece are preferably equipped with a heating apparatus. The heating apparatus can be used to locally adjust the temperature in a controlled manner. The heating apparatus at the first outer face, especially in the second section, can especially preheat the second input gas. The connecting piece may be heated in order to promote the chemical reaction.

The first reactor is preferably manufactured from a material impervious to the electromagnetic waves, especially steel, bronze or aluminum. The second reactor, especially including the inner tube, and/or the connecting piece are preferably manufactured from a material of high thermal stability, especially graphite, quartz glass or a metal such as tungsten or molybdenum. The material of high thermal stability is stable in particular up to a temperature of 2000° C.

The first reactor is preferably a closed vessel except for the bushing for the connecting piece. In particular, both first end faces of the first reactor are preferably closed, where one of the first end faces has the connecting piece.

The chemical reaction is preferably an endothermic reaction, i.e. one that requires supply of energy. An energy source used is the plasma which is generated by means of the electromagnetic waves in the first reactor. The chemical reaction preferably takes place in the plasma phase and/or gas phase. The plasma phase and/or gas phase, or the reactants, may comprise gases and/or vaporous components. A solid material is preferably involved in the chemical reaction. What are meant in particular by reactants, which can also be referred to as starting materials or educts, are substances that are fed to the first reactor and/or the second reactor, preferably the first reactor, and chemically converted therein. The solid material is especially formed or used as a reactant, i.e. is converted. The solid material preferably contains carbon, and is further preferably carbon, i.e. consists of carbon. The carbon is especially atomic carbon, which is also referred to as soot, industrial carbon black or carbon black.

The chemical reaction is especially selected from the group consisting of pyrolysis of hydrocarbons, especially of methane, production of acetylene, reforming of hydrocarbons, especially methane, pyrolysis of hydrogen sulfide, pyrolysis of ammonia, and hydrogasification of carbon.

The pressure in the first reactor may be ambient pressure, elevated pressure or a reduced pressure. The reaction temperature is preferably more than 2300° C. The reaction is primarily determined kinetically, such that parameters of relevance are not only temperature but also those such as pressure or dwell time. The temperature in the first reactor, especially in the plasma, is preferably within a range from 2000° C. to 5000° C. The pressure in the first reactor, in the connecting piece and/or in the second reactor is preferably 1 Pa to 1 MPa absolute, more preferably 0.005 MPa to 0.2 MPa absolute, i.e. 50 mbar to 2 bar absolute. The dwell time is especially determined via the dimensions of the second reactor and is preferably matched to the reaction kinetics. The dwell time in the first reactor and/or in the second reactor is preferably less than 1 second in each case. Further preferably, the dwell time in the first reactor and/or in the second reactor is within a range from 10 milliseconds to 500 milliseconds in each case.

The chemical reaction may be the plasma-induced pyrolysis of hydrocarbons, especially methane. In a further embodiment, methane can be reformed using carbon dioxide and/or water vapor. In addition, the chemical reaction may be the pyrolysis of hydrogen sulfide or ammonia.

Preferably, the first input gas is fed to the first reactor at the at least one inlet of the first section, and the second input gas at the at least one inlet of the second section, and the product stream is preferably withdrawn from the second reactor at the outlet, where the first section is disposed on an opposite side of the window from the connecting piece. The first input gas and the second input gas are preferably each conducted tangentially into the first reactor and mixed in the first reactor, and form a plasma under the action of the electromagnetic waves. The feeding, mixing and plasma formation are preferably effected in the sequence specified. In particular, the first input gas and the second input gas are first mixed and then exposed to the electromagnetic waves in a mixture.

The first input gas preferably contains hydrocarbons, especially methane, and/or other reactants. In addition, the first input gas may comprise further gases such as hydrogen, inert gases and/or water vapor. In addition, the first input gas may comprise a solid material such as carbon. The reactants are preferably added to the first input gas in pure form, especially in a concentration of more than 99% by volume. The first input gas may consist to an extent of more than 99% by volume of reactants, especially hydrocarbons, for example methane. The first input gas preferably comprises biogas, especially a mixture comprising methane and carbon dioxide, offgas such as flare gas, especially from refineries, and/or some other mixture of hydrocarbons. The addition of mixtures of reactants is also possible depending on the process. The first input gas may comprise and especially consist of, for example, 50% to 60% by volume of methane and 40% to 50% by volume of carbon dioxide, based on the overall first input gas, especially when biogas is used.

In addition, the first input gas may comprise and especially consist of, for example, 3% to 95% by volume of hydrocarbons and 5% to 97% by volume of hydrogen, based on the overall first input gas, especially when offgas is used. The first input gas here typically comprises 40% by volume of C₁₋₂ hydrocarbons having one or two carbon atoms, 9% by volume of C₃₊ hydrocarbons having three or more carbon atoms, 3% by volume of carbon dioxide, 1% by volume of nitrogen and 47% by volume of hydrogen.

The first input gas further preferably comprises methane, with or without hydrogen and/or carbon dioxide. The first input gas is preferably at a temperature of not more than 600° C., especially in the case of hydrocarbons such as methane, in order to avoid soot formation. The temperature of the first input gas is preferably within a range from 20° C. to 600° C., more preferably within a range from 300° C. to 1500° C.

The second input gas preferably comprises an inert gas, water vapor and/or hydrogen, with or without hydrocarbons, especially methane, or a mixture of various reactants, especially various hydrocarbons. The hydrocarbons are especially present in only a small amount or low concentration in the second input gas. In particular, the concentration of hydrocarbons in the second input gas is lower than in the first input gas. Accordingly, the first input gas is preferably diluted by the second input gas in the first reactor.

The concentration of hydrocarbons, including methane in particular, in the sum total of the input gases, i.e. the first input gas and the second input gas, is preferably 50% by volume or less. This avoids the unwanted formation in this phase of the process of solid reaction products such as carbon, which would hinder the formation of the plasma.

The volume ratio of the first input gas to the second input gas, especially of the first input gas stream to the second input gas stream, is preferably less than 1. The volume ratio of the two input streams influences the position of the mixing zone and avoids a position of the mixing zone at the window which is transparent to the electromagnetic waves. Addition of the first input gas and the second input gas in a ratio of 1, where the first input gas contains pure reactants, for example pure methane, results, for example, in a mixture containing 50% by volume of the reactant or methane in the first reactor. In a further example, the second input gas may be used for enrichment with carbon dioxide, in order to achieve a stoichiometric ratio of 1:1 and a concentration of 50% by volume of methane when biogas is converted to synthesis gas, for example.

The inert gas comprises or consists of preferably nitrogen and/or argon.

The product stream preferably comprises the solid material, especially carbon. The product stream may be at least partly recycled, especially into the first reactor. This can increase the yield. In the case of recycling, any solid material present in the product stream may be separated from the gas phase. For example, in the case of methane pyrolysis, pulverulent carbon can be separated off in the recycling. This counteracts any hindrance of plasma formation. In the hydrogasification of carbon, for example, preference is given to dispensing with any removal of solids in the recycling. In the case of hydrogasification, the carbon is a reactant that cannot be removed from the conversion. The solid material can be removed using filters and/or cyclones. The product stream can be recycled as part of the first input gas and/or of the second input gas. The choice depends on the reaction conducted in the reactor. In the case of hydrogasification of carbon, the recycling of the product stream is preferably implemented, for example, via the first input gas, in order to optimize plasma formation.

In a preferred embodiment, heat is transferred from the product stream to the first input gas and/or the second input gas. For this purpose, in particular, the product stream is fed to a first space in a heat exchanger and the first input gas, especially a first input gas stream, and/or the second input gas, especially a second input gas stream, is fed to a second space in the heat exchanger, such that heat transfer takes place from the product stream to the first input gas and/or the second input gas. This achieves heat recovery which improves energy management with regard to the reactor and increases the efficiency of the method. The power consumption required for the plasma is reduced. The heat transfer is preferably connected upstream of any possible removal of solids.

The third input gas preferably comprises hydrogen and/or nitrogen.

The method may be used for removal and immobilization of carbon dioxide (CO₂) and/or an offgas from the atmosphere and may additionally comprise the following steps:
a) converting the carbon dioxide from the atmosphere and/or from an offgas to biomass by means of photosynthesis, preferably in agricultural areas, in particular in a greenhouse,
b) conducting a biogas reaction in which the biomass produced in step a) is converted to biogas comprising methane and carbon dioxide, in particular in a biogas reactor,
c) separating the methane from the biogas obtained,
d) cracking the methane to carbon and hydrogen, with the carbon obtained in solid form,
e) collecting the carbon obtained and
f) landfilling the carbon obtained.

Steps a) to f) comprise three biological or chemical reactions in order to remove CO₂ from the air. These are photosynthesis in step a), the biogas reaction, in particular an anaerobic fermentation, in step b), and the cracking of the methane, in particular a pyrolysis of methane, in step d).

Step d) is preferably conducted in the apparatus of the invention.

Step a) for production of biomass may take place in an agricultural process, i.e. in an area used for agriculture, and/or in a greenhouse.

Biomass is defined in particular as substances and mixtures thereof that are produced by photosynthesis. These are also referred to as primary biomass. Biomass also refers to substances and mixtures thereof that have been formed as a result of the use of the primary biomass and have retained biogenic character. In particular, the biomass may comprise straw, forestry wastes, farm fertilizers, food wastes and/or municipal wastes.

The offgas from which the CO₂ is possibly converted to biomass in step a) may, for example, be an offgas formed in the combustion of fossil fuels in a power plant. The offgas may, for example, also be a by-product which is formed in an industrial production process, or the offgas may be a production gas which is produced in the production of fossil fuels such as coal, oil or natural gas. The offgas fed to step a) is in particular a mixture comprising CO₂. The offgas may additionally comprise, as further components, at least one inert gas such as nitrogen or argon and possibly water vapor.

Preferably, the offgas used in step a) originates at least in part from the use of the hydrogen produced in step d) or of the biogas formed in step b), in particular the methane. The offgas is optionally desulfurized in a pretreatment, freed of the other impurities and/or dedusted. Further preferably, the offgas used in step a) consists exclusively of offgas which is recycled within the process. More preferably, the offgas used in step a) originates exclusively from the biogas produced in step b), in particular from the use of the hydrogen produced in step d). Accordingly, the offgas may originate from the biogas directly or indirectly, i.e. after subsequent cracking of the methane.

The biogas reaction in step b) especially comprises an anerobic fermentation reaction. In anerobic fermentation the biomass is converted to biogas, i.e. a mixture of predominantly methane and carbon dioxide. The biogas produced in step b) preferably contains a total of at least 90% by volume, further preferably at least 95% by volume, of methane and CO₂, based on the overall biogas.

Biomass used in step b) is preferably plants, in particular C₃ plants, biowastes, farm fertilizers such as manure and/or slurry, and/or municipal wastes. The biomass produced in step a) can be used directly in step b). For instance, plants produced in step a) can be supplied directly to the biogas reaction in step b). Alternatively, the biomass produced in step a) can first be subjected at least in part to utilization or conversion, such that takes the form of biowastes, municipal wastes or fertilizers in step b).

The biogas reaction is preferably performed in a closed vessel, also referred to as a fermenter. The biomass may be comminuted and optionally sorted prior to conduction of the biogas reaction. The biomass may be supplied continuously to the fermenter. The residence time of the biomass in the fermenter is preferably more than one day. The produced biogas accumulates in particular in an upper region of the fermenter above a liquid phase and solid phase. The biogas preferably contains at least 40% by volume, further preferably 50% by volume to 75% by volume, especially 62% by volume to 75% by volume, of methane, based on the overall biogas. The biogas further comprises CO₂, with or without water vapor, hydrogen sulfide and/or ammonia.

The biogas produced comprises CO₂, which can be separated off in a workup of the biogas, in particular in a CO₂ separation apparatus, and fed back to the photosynthesis. Step c) can accordingly also be referred to as workup of the biogas. The carbon dioxide can be separated off using polyimide hollow fiber membranes for example. Preference is given to returning the CO₂ from the biogas obtained to step a) after the methane has been separated off in step c). In addition, the CO₂ from the biogas obtained can be liquefied after methane has been separated off in step c) and/or the CO₂ from the biogas obtained can be converted to a hydrocarbon mixture after methane has been separated off in step c), in particular fed to a Fischer-Tropsch apparatus.

Preferably, in a step g), CO₂ from the biogas, especially after methane has been separated off in step c), is reacted with hydrogen produced in step d) to give a hydrocarbon mixture, where the hydrocarbon mixture especially comprises kerosene, gasoline and/or waxes.

The methane is preferably separated off in step c) by physical means, in particular by means of condensation, adsorption and/or a membrane process. The separation of the methane from the biogas obtained in step c) is preferably conducted continuously.

In particular, pressure swing adsorption, temperature-vacuum adsorption, chemical adsorption, membrane separation, compressed gas scrubbing processes and/or concentration swing adsorption are used for separation of the methane.

After the separation in step c), the stream of matter of the separated methane preferably contains more than 95% by volume of methane and further preferably not more than 1% by volume of CO₂.

The energy required for operation of the apparatus of the invention is preferably provided in the form of power from renewable sources such as wind, biogas and/or photovoltaics which in particular cannot be accepted at the time in the power grid. Alternatively, a portion of the biogas or of the obtained hydrogen can be used to provide the energy needed for the cracking.

A filter in which the carbon is separated from the hydrogen is preferably connected downstream of step d). The conversion of the methane to hydrogen and carbon is preferably effected with a yield of about 96% to 97%.

Preferably, at least a portion of the hydrogen produced in step d)/of the biogas produced in step b) will be used as an energy source for cracking the methane.

The hydrogen produced in step d) is preferably used at least in part as a starting material for syntheses in the chemical industry, as an energy carrier for the generation of electric power, heat, optionally refrigeration, and/or as a fuel for vehicles.

The carbon is preferably collected, transported to a landfill site and subjected to long-term storage or final storage. The carbon obtained in step d) is preferably completely landfilled.

The carbon obtained in step d) is further preferably mixed with other components, in particular further solids, prior to landfilling in step f), such that use for energy purposes is no longer possible, in order to ultimately immobilize the carbon and be able to store it particularly securely. The further solids used may include, for example, sand, loam, gravel, construction rubble, slags, stones, waste, in particular from industrial dismantling, or a combination of two or more of these materials. Accordingly, the carbon obtained in step d) is preferably immobilized, and long-term immobilization is ensured by the mixing of the carbon obtained with a further solid. The solids mixture formed, which comprises the carbon obtained with at least one further solid, is stored in particular geologically and for the long term in a carbon sink, such as a mine.

The landfilling in step f) preferably extends to at least 30 years, further preferably at least 50 years.

In addition, the method or the apparatus can be used for storage and for transport of electrical energy. In a preferred embodiment, the apparatus is used in a storage and/or transport method comprising the following steps; in particular, the method of the invention additionally comprises the following steps:
i. producing methane from water and carbon using electrical energy,
ii. storing the methane,
iii. cracking the methane to hydrogen and carbon, where the hydrogen is used for energy generation and/or as reactant in a chemical reaction, or energy generation by converting the methane to carbon and water in a cyclical bromination-oxidation process,
   wherein the carbon obtained in methane cracking or the cyclical bromination-oxidation process in step iii. is collected and, in a new process procedure, is used for the production of methane in step i., giving rise to a closed carbon circuit.

In particular, the production of methane in step i. and/or the cracking of methane in step iii. are performed in the apparatus of the invention.

The carbon preferably serves as carrier for hydrogen in the storage and/or transport method. In particular, the carbon is not used here as fuel for energy generation. Energy generation in this connection especially means power generation and/or the use of the hydrogen obtained by the cracking of the methane in step iii. for further energy applications, such as heating, cooling or driving of vehicles such as cars, trucks, trains or ships.

In step i., synthesis gas is preferably first produced by reacting the carbon, especially in pulverulent form, with water vapor to form a mixture comprising carbon monoxide and hydrogen. The energy required for the purpose is preferably generated by means of renewable sources. The synthesis gas is then preferably catalytically converted to methane with hydrogen that is especially produced by electrolysis from water and power from renewable sources.

### Advantages of the Invention

The apparatus of the invention or the method of the invention allows introduction of the first input gas and the second input gas that form the reaction mixture independently into the first reactor. The feeding of the second input gas on the opposite side with respect to the window stabilizes the reaction system.

The separate supply of the first input gas and the second input gas has the advantage that the reaction takes place only after the two streams have been mixed with one another, and this is done in a controlled manner in a mixing zone in which the two flows of the first input gas and of the second input gas in vortex form meet one another, are mixed vigorously and form an inner vortexlike flow. This ensures that a homogeneous mixture of the reactants is converted to plasma, and no bypass effects occur.

The plasma and products formed in the first chemical reaction are stabilized in a middle region of the first reactor, i.e. at a distance from the reactor wall. This is particularly advantageous when the reaction products are at least partly in solid form and have a tendency to be deposited on the inner wall of the reactor, which can result in local overheating of the reactor wall and in disruption of the reactor functions. The formation of the vortex flow close to the reactor wall prevents these adverse effects.

The chemical reaction is continued virtually adiabatically in the connecting piece, and it is ensured that the plasma reaches the second reactor.

The second reactor extends the dwell time in the cascade system, in order to conclude the chemical reaction. There is preferably no electromagnetic field in the second reactor. The optimal dwell time can be adjusted over the length of the first reactor and the second reactor, and permits achievement of the desired reaction results.

The vortex flows of the reaction mixture achieve flow conditions similar to those of an ideal tubular reactor. It is thus possible to conduct the reaction adiabatically, since axial mixing within the reactor is minimized.

The arrangement of the inner tube, especially in coaxial form, in the second reactor counteracts the deposition of solid reaction products on the walls of the second reactor, since the flow rate in the outer gas space, i.e. between inner tube and inner wall of the second reactor, is increased.

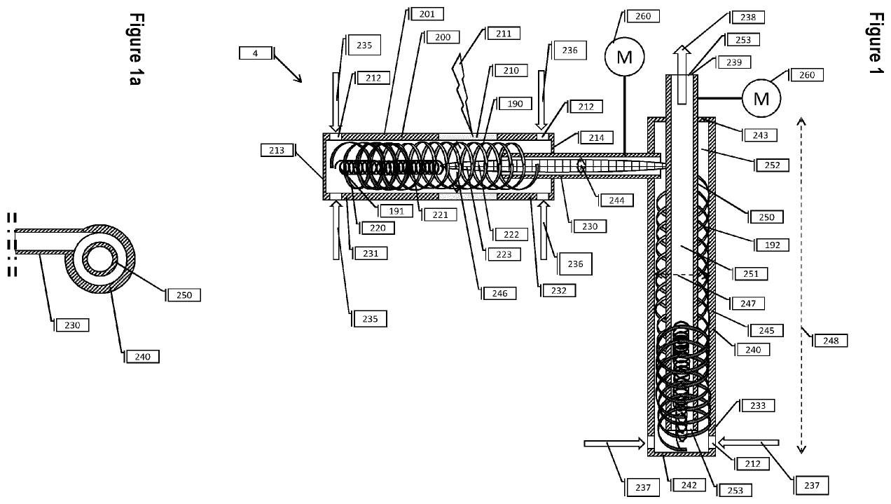

The invention is described in detail with reference to the drawings that follow (FIGS. 1-5), the list of reference numerals and the claims, and also the examples.

The figures show:

FIG. 1 a first embodiment of the apparatus of the invention,

FIG. 1a a cross-sectional view of the first embodiment of the apparatus,

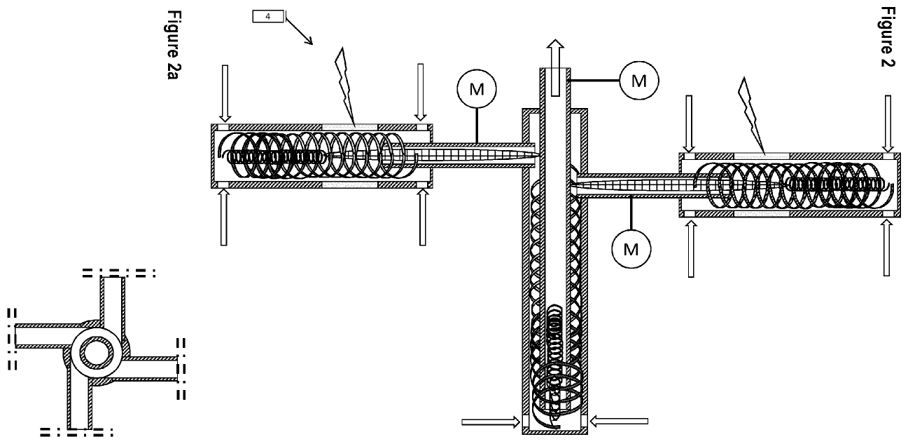

FIG. 2 a second embodiment of the apparatus of the invention,

FIG. 2a a cross-sectional view of the second embodiment of the apparatus of the invention,

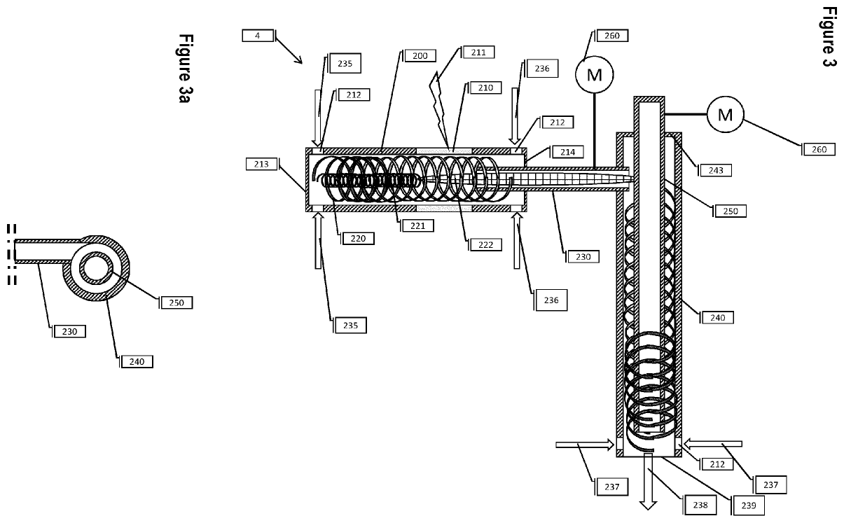

FIG. 3 a third embodiment of the apparatus of the invention,

FIG. 3a a cross-sectional view of the third embodiment of the apparatus of the invention,

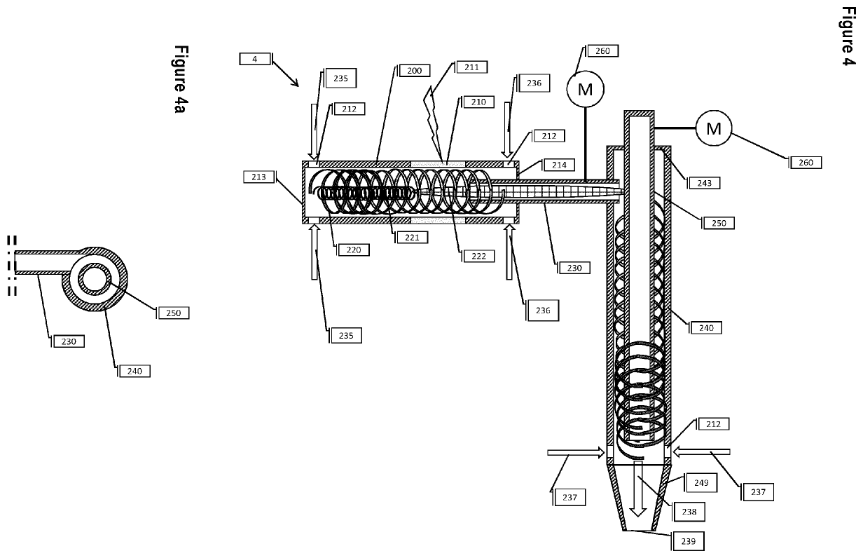

FIG. 4 a fourth embodiment of the apparatus of the invention,

FIG. 4a a cross-sectional view of the fourth embodiment of the apparatus of the invention,

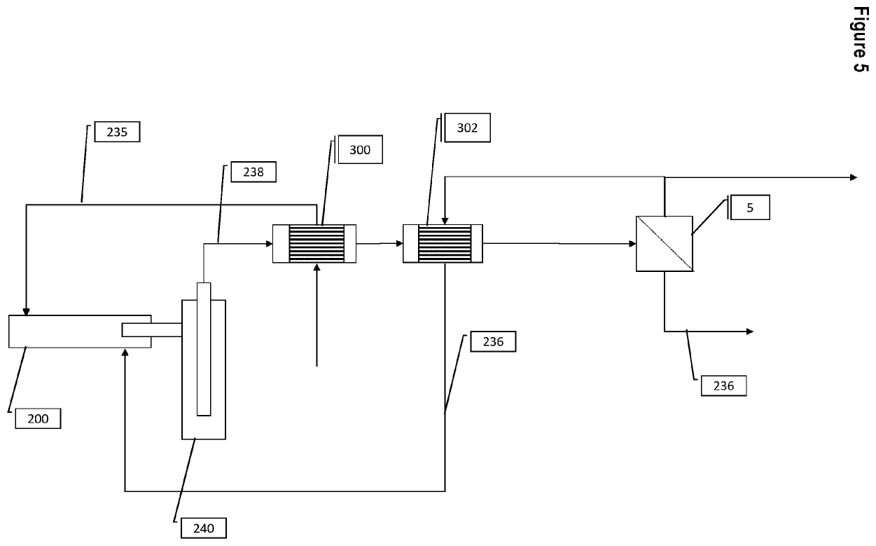

FIG. 5 a scheme of the optional heat recovery and solids removal,

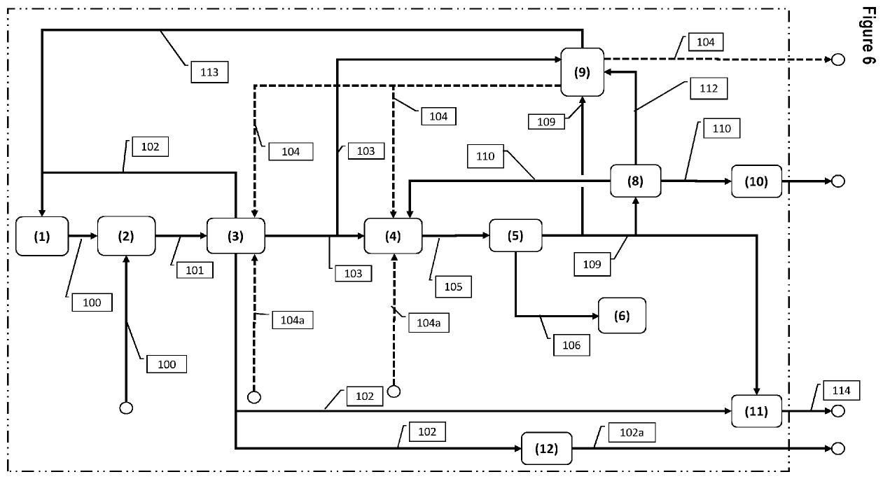

FIG. 6 a scheme of a method in which the inventive apparatus 4 is used and

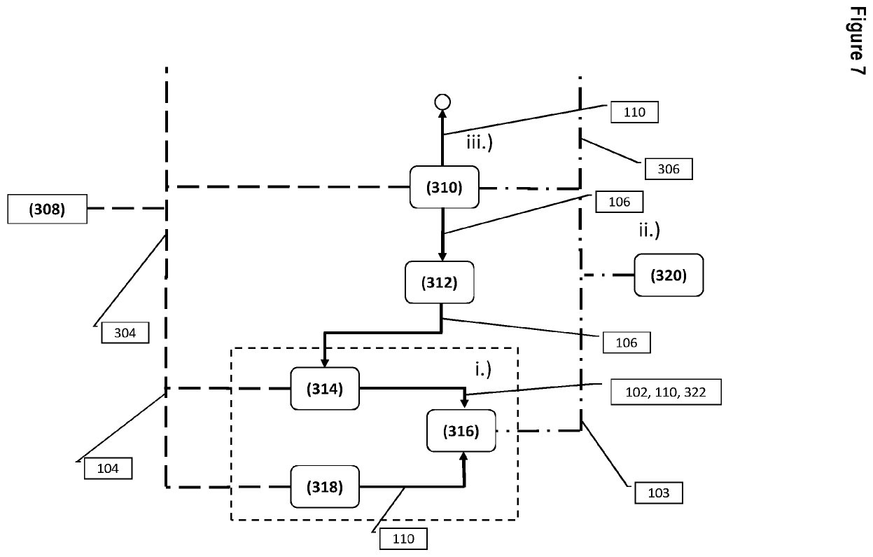

FIG. 7 a scheme of a further method in which the inventive apparatus 4 is used.

FIG. 1 shows a first embodiment of the inventive apparatus 4 in longitudinal section. The apparatus 4 has a first reactor 200 and a second reactor 240, which are fluidically connected to one another by a connecting piece 230.

The first reactor 200 is bounded by a first outer face 201, and first end faces 213, 214. By means of opposite inlets 212 in a first section 231 of the first outer face 201, a first input gas 235 is fed tangentially into the first reactor 200, forming a first vortex flow 220. The first vortex flow 220 meets a second vortex flow 190 of a second input gas 236, flowing in the opposite direction, which is likewise fed tangentially into the first reactor 200 at two inlets 212 in a second section 232 of the first outer face 201.

The first vortex flow 220 and the second vortex flow 190 meet in a mixing zone 221 in the first reactor 200, where the first input gas 235 and a second input gas 236 are mixed with one another and form a third vortex flow 191 with which the input gas mixture formed is exposed to electromagnetic waves 211 at a window 210 in the first reactor 200, so as to form a plasma 223 that leaves the first reactor 200 via the connecting piece 230.

The plasma 223 is indicated in a plasma zone 222 at the window 210 and is supported by the second vortex flow 190 that surrounds the plasma 223 in the vicinity of the wall of the first reactor 200. Then the plasma 223 is introduced tangentially into the second reactor 240. The second reactor 240 has an inner tube 250, so as to form an inner gas space 251 and an outer gas space 252. The plasma 223 enters the outer gas space 252 and forms a fourth vortex flow 192 around the inner tube 250.

The second reactor 240 is bounded by a second outer face 245, and second end faces 242, 243. At one of the second end faces 242 at a distance from the connecting piece 230, inlets 212 are provided in a third section 233, through which a third input gas 237 is fed for cooling.

A product stream 238 leaves the second reactor 240 through an outlet 239 at an open end 253 of the inner tube 250.

The connecting piece 230 has a constant diameter 244. The first reactor 200 has a constant first reactor diameter 246, and the second reactor 240 a constant second reactor diameter 247. In addition, the second reactor 240 has a total length 248.

In particular, the formation of the first vortex flow 220 and the second vortex flow 190 that meet in countercurrent outside the window 210 in the first reactor 200 achieves a homogeneous input gas mixture which is then transferred into the plasma 223 at the window 210 for the chemical reaction to proceed. In addition, the first vortex flow 220 and the second vortex flow 190 prevent solid deposits on the first outer face 201 and especially on the window 210.

The inner tube 250 in the second reactor 240 ensures a high flow rate in the outer gas space 252, such that solid deposits are avoided in the second reactor 240 as well. Solid deposits are additionally prevented by vibration apparatuses 260 on the connecting piece 230 and the inner tube 250.

FIG. 1a shows a cross-sectional view of the second reactor 240 with the inner tube 250 and the connecting piece 230 in the first embodiment of the apparatus 4. In this cross-sectional view, the connection of the connecting piece 230 in tangential alignment to the second reactor 240 is apparent.

FIG. 2 shows a second embodiment of the inventive apparatus 4 that corresponds essentially to the first embodiment according to FIG. 1, except that four first reactors 200 are connected to the second reactor 240 by one connecting piece 230 each.

FIG. 2a shows a cross-sectional view of the second reactor 240 in the second embodiment with four connecting pieces 230 in tangential arrangement.

FIG. 3 shows a third embodiment of the inventive apparatus 4 that differs from the first embodiment according to FIG. 1 in the design of the second reactor 240. In the second embodiment, the inner tube 250 has an open end 253 and a closed end 254, where the open end 253 is disposed at the second end face 242 further away from the connecting piece 230, which is also the location, in this embodiment, of the outlet 239 where the product gas stream 238 is withdrawn. In the third embodiment, product stream does not flow through the inner tube 250 in the inner gas space 51. There is only the fourth vortex flow 192 around the inner tube 250.

FIG. 3a shows a cross-sectional view of the second reactor 240 with the connecting piece 230 in the third embodiment.

FIG. 4 shows a fourth embodiment of the inventive apparatus 4 that differs from the third embodiment according to FIG. 3 in the configuration of the outlet 239 as one conical end 249 of the second reactor 240.

FIG. 4a shows a cross-sectional view of the second reactor 240 with the connecting piece 230 in the fourth embodiment.

FIG. 5 shows a scheme of the optional heat recovery and solids removal. The second reactor 240 has a downstream first heat exchanger 300 and second heat exchanger 302, where the second heat exchanger 302 is connected to a filter 5 for solids removal. The product stream 238 which is withdrawn from the second reactor 240 is cooled. In the first heat exchanger 300, heat is first released to a first input gas 235, and, in the second heat exchanger 302, heat is transferred from the product stream 238 to a second input gas 236. The first input gas 235 is fed to the first reactor 100 by the first heat exchanger 300, and the second input gas 236 by the second heat exchanger 302. Downstream of the second heat exchanger, the product stream 238 is conducted through the filter 5, where a solid material, especially carbon 106, is separated off. The remaining gaseous product gas is partly recycled into the first reactor 200 as second input gas 236.

FIG. 6 shows a scheme of a method in which the inventive apparatus 4 is used as reactor.

In a step a), biomass 100 is produced by photosynthesis from CO₂ 102. This is done, for example, by growing plants in a field or, alternatively or additionally, by cultivating plants in a greenhouse 1.

To increase plant growth, the air in the greenhouse 1 can be enriched with CO₂ 102 and heated. The CO₂ 102 can be supplied separately and/or as part of an offgas 113 to the greenhouse 1. The CO₂ 102 is preferably recycled from further steps of the process, in particular from a workup 3 of biogas 101 produced in the process and as part of the offgas 113 from a combined heat and power (CHP) plant 9.

The biomass 100 is fermented in a step b) of the process, in particular with an anaerobic bacterial reaction, to give biogas 101 comprising methane 103 and CO₂ 102. The reaction is conducted in particular continuously in a mixing vessel constituting a biogas reactor 2.

In a step c) of the process, the biogas 101 is supplied to a workup 3, wherein methane 103 in the form of a methane-rich stream is separated from a stream containing CO₂ 102. The CO₂ 102, for example the entire CO₂-comprising stream 102, can be recycled into the greenhouse 1 in order to enrich the air in the greenhouse 1 with CO₂ 102. Alternatively or in addition to the recycling of the CO₂ 102 from the workup 3 into the greenhouse 1, the CO₂ 102 can be conducted to a Fischer-Tropsch apparatus 11.

In a step d) of the process, the methane 103 from the workup 3 is fed to the inventive apparatus 4. In the apparatus 4, methane 103 is cracked into its constituents: carbon in solid form 106 and hydrogen 110.

The conversion of the methane 103 is incomplete, and other hydrocarbons are likewise obtained in small amounts. At the outlet of the reactor 4, a product gas mixture 105 is formed, comprising gaseous methane 103, hydrogen 110 and other hydrocarbons, and also carbon in solid form 106.

The apparatus 4 is preferably operated using energy such as electrical power 104 from renewable sources, in particular from wind energy and photovoltaics. The electrical power 104 may alternatively or additionally be generated in the CHP plant 9.

Connected downstream of the apparatus 4 is a filter 5 in which the separation of the solid 106 from a gas phase 109, which contains hydrogen 110, takes place.

The gas phase 109 may optionally be intermediately stored and supplied as fuel to the integrated CHP plant 9. Furthermore, the electric current 104 generated in the CHP plant can be fed into the public power grid in order to stabilize the grid.

Alternatively, the hydrogen 110 can be separated from tail gas 112 present in the gas phase 109 in a pressure swing adsorption system or by means of membranes 8. A portion of the hydrogen 110 can be recycled into the reactor 4 and a further portion can be used further as product.

Alternatively or additionally, the gas phase 109 can be fed to the Fischer-Tropsch apparatus 11. The gas phase 109 together with CO₂ 102 originating from the workup 3 can be converted here to a hydrocarbon mixture 114. The hydrocarbon mixture 114 preferably contains kerosene, gasoline, waxes and mixtures thereof.

The tail gas 112 used after the separation from the gas phase 109 and serving as fuel can optionally be stored intermediately and optionally supplied to the CHP plant 9. As an alternative to an external power source, the electric current 104 generated in the CHP plant 9 can be used for the cracking of the methane 103 in the apparatus 4. Furthermore, the offgases 113 produced in the CHP plant 9 are preferably supplied to the greenhouse 1, in particular together with the heat 115 generated therein.

FIG. 7 shows a scheme of a further method in which the inventive apparatus 4 is used. What is shown in schematic form is the storage and/or transport method that connects a power grid 304 and a gas grid 306 to one another by multiple method steps. The power grid 304 is fed from renewable energies 308, for example based on wind energy or solar energy. Electrical power 104 is used in a step i. in a hydrogasification 314 of carbon 106 and an electrolysis 318 for production of methane 103. For this purpose, carbon dioxide 102, carbon monoxide 322 and hydrogen 110 are fed to a methanation 316. Methane produced, in a step ii., is stored in a gas storage means 320 and, in a step iii., cracked in a hydrogen generator 310 to hydrogen 110 and carbon 106. The carbon 106 is intermediately stored in a carbon storage means 312. The hydrogen can be used to generate heat, in transportation or for power generation, or be converted in the chemical industry. The inventive apparatus 4 is used in the hydrogasification 314 and in the hydrogen generator 310.

### EXAMPLES

### Working Example 1: Pyrolysis of Methane

The pyrolysis of methane, which can be used in the form of natural gas, synthetic natural gas (SNG) or biomethane, is described by the following reaction equation:

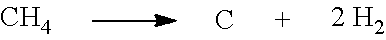

Methane and hydrogen, which is also referred to here as plasma gas, are used. A portion of the hydrogen is fed to the second section of the apparatus; the remaining hydrogen is fed together with methane to the first section of the first reactor, in each case via two inlets. The volume ratio of the streams in the first section and in the second section is 1:1, such that the mixing zone of the two streams is in the middle of the first reactor.

The hydrogen fed to the second section forms a thin layer in the form of a vortex at the reactor wall and at the window that is transparent to electromagnetic waves. This prevents carbon particles formed in the reaction from being deposited on the wall of the first reactor. Deposition thereof would have the effect that electromagnetic waves are absorbed, the window overheats and the first reactor is destroyed.

The reaction takes place first in the mixing zone and then in the plasma.

The carbon formed leaves the first reactor in solid form together with the product gas stream, i.e. the gas phase, via the connecting piece. The diameter of the connecting piece has such dimensions that the flow rate of the biphasic flow is at least 20 m/s.

The plasma zone and connecting piece form a virtually (quasi-) adiabatic tubular reactor. The energy introduced by means of the electromagnetic waves is absorbed and consumed by the chemical reaction.

### Working Example 2: Acetylene Production

If there is a reduced pressure, especially a vacuum, within a range from 50 to 100 mbar absolute in the first reactor, the formation of acetylene from methane in the plasma in accordance with the following reaction equation is promoted:

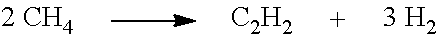

Methane is added at the first inlet of the first reactor, hydrogen at the second inlet. Proceeding from the second section, two near-wall vortex flows of methane and hydrogen are correspondingly formed, which are first conducted into the middle of the first reactor. The two streams are mixed, forming plasma in the region of the window. After the plasma has been formed, the temperature is in the range from 3000 to 3500° C.

The ionization and fragmentation of the input gases under reduced pressure leads to formation of acetylene. The reaction according to working example 1, although suppressed by the reduced pressure, likewise takes place to a reduced degree, such that carbon in solid form is formed in small amounts. Here too, the deposition of the solids material is counteracted by the conduction of gas in vortex form.

In order to avoid breakdown of the acetylene formed in the case of only gradually falling temperature, which would form solid carbon and hydrogen, the product gas is quenched, with supply of a cold third input gas to the second reactor in the third section. The third input gas used is hydrogen.

### Working Example 3: Dry Reforming of Biogas

The first and second input gases used are biogas. The concentration of methane in the biogas varies significantly and is between 40% and 75% by volume. The reaction runs according to the stoichiometric equation:

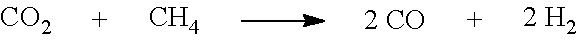

In order to enable an equimolar ratio between the two reactants methane and carbon dioxide, depending on the methane concentration, methane is added in the first inlet or carbon dioxide in the second inlet. For example, in the case of a methane concentration of 70% by volume in the biogas, the first input gas consists of one part by volume biogas, and the second input gas of one part biogas and 0.8 part by volume CO₂.

The plasma is stabilized in the middle of the first reactor and in the connecting piece, and the reforming reaction is continued adiabatically in the second reactor.

The reforming reaction proceeds in two steps. First of all, methane is cracked in the plasma to its constituents: hydrogen and atomic carbon/soot. In a second step, carbon reacts with carbon dioxide according to the Boudouard principle to give carbon monoxide:

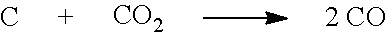

The high temperature in the plasma promotes shifting of the reaction equilibrium of carbon and carbon dioxide in favor of carbon monoxide formation. The flows in vortex form in the first reactor and in the second reactor prevent deposits of solids on the walls, such that they remain in the gas phase and react with the carbon dioxide.

In order to prevent the side reaction

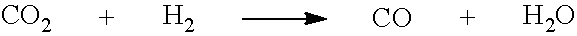

which is unwanted here, the product gas stream is quenched by feeding cold hydrogen gas into the second reactor as third input gas in the third section.

### Working Example 4: Methane-Steam Reforming

Methane is reacted with water vapor to give synthesis gas in plasma:

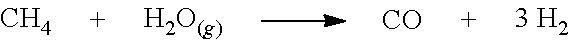

Methane is fed into the first reactor as the first input gas in the first section via two feeds. Water vapor is introduced into the first reactor as the second input gas in the second section via two feeds. The water vapor forms a flow in the form of a vortex close to the wall of the first reactor and stabilizes the plasma. The water vapor and the methane are mixed when the two vortices meet and form an inner vortex that forms a methane-water vapor plasma at the window. The input gas mixture is ionized there at high temperatures and breaks down into smaller fragments, i.e. free radicals. The plasma is transferred to the second reactor via the connecting piece, and the free radicals form the reaction products that are withdrawn as product stream.

### Working Example 5: Hydrogasification of Carbon

The reaction of pulverulent carbon with water vapor to give synthesis gas is performed using plasma:

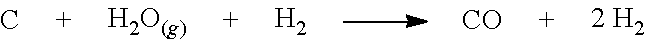

The reaction is endothermic. In order to promote plasma formation, hydrogen is additionally used as plasma gas. The first input gas fed to the first reactor in the first section is a mixture of carbon and water vapor. Water vapor is used in excess, compared to the stoichiometric ratio of the reactants. Hydrogen is fed in as the second input gas in the second section. The carbon is also added in the first section of the first reactor. This ensures that the pulverulent carbon does not impair plasma formation.

### LIST OF REFERENCE NUMERALS

1 greenhouse
2 biogas reactor
3 workup
4 apparatus
5 filter
8 membranes
9 combined heat and power plant
11 Fischer-Tropsch apparatus
100 biomass
101 biogas
102 CO₂
103 methane
104 electrical power
105 product gas mixture
106 solid material (carbon)
109 gas phase
110 hydrogen
112 tail gas
113 offgas
114 hydrocarbon mixture
115 heat
190 second vortex flow
191 third vortex flow
192 fourth vortex flow
200 first reactor
201 first outer face
210 window
211 electromagnetic waves
212 inlet
213, 214 first end faces
220 first vortex flow
221 mixing zone
222 plasma zone
223 plasma
230 connecting piece
231 first section
232 second section
233 third section
235 first input gas
236 second input gas
237 third input gas
238 product stream
239 outlet
240 second reactor
242, 243 second end faces
244 diameter of the connecting piece
245 second outer face
246 first reactor diameter
247 second reactor diameter
248 total length
249 conical end
250 inner tube
251 inner gas space
252 outer gas space
253 open end
254 closed end
260 vibration apparatus
300 first heat exchanger
302 second heat exchanger
304 power grid
306 gas grid
308 renewable energy
310 hydrogen generator
312 carbon storage means
314 hydrogasification
316 methanation
318 electrolysis
320 gas storage means
322 carbon monoxide

1. An apparatus for conducting a chemical reaction in a plasma, the apparatus comprising:
a source for generation of electromagnetic waves;
at least one first reactor;
at least one connecting piece; and
a second reactor,
wherein the at least one first reactor, the at least one connecting piece, and the second reactor are each configured as tubes,
wherein the at least one connecting piece has a smaller diameter than the at least one first reactor, and
wherein the source for generation of electromagnetic waves is disposed on the at least one first reactor,
wherein the at least one first reactor has a first outer face and first end faces, and the second reactor has a second outer face and second end faces,
wherein the second reactor has an inner tube disposed at least partly within the second reactor to form an inner gas space and an outer gas space that are separated from one another at the second end face of the second reactor that is closer to the at least one connecting piece,
wherein the at least one connecting piece fluidically interconnects the at least one first reactor and the second reactor,
wherein the at least one connecting piece exits from the at least one first reactor at one of the first end faces and opens into the outer gas space of the second reactor at the second outer face,
wherein the first outer face of the at least one first reactor has a first section having at least one inlet for supply of a first input gas, a second section having at least one inlet for supply of a second input gas, and a window which is transparent to the electromagnetic waves,
wherein the at least one inlet for supply of the first input gas and the at least one inlet for supply of the second input gas are aligned tangentially to the first outer face of the at least one first reactor,
wherein the first section and the second section are in an axially offset arrangement,
wherein the second section lies closer than the first section to the first end face from which the at least one connecting piece exits from the at least one first reactor,
wherein the window is disposed between the first section and the second section,
wherein the second reactor has an outlet for removal of a product stream,
wherein the second outer face of the second reactor has a third section having at least one inlet for supply of a third input gas, and
wherein the third section is disposed at one of the second end faces of the second reactor that is further away from the at least one connecting piece.

2. The apparatus as claimed in of claim 1, wherein the at least one connecting piece extends within the at least one first reactor via the at least one inlet of the second section up to no further than the window.

3. The apparatus of claim 1, wherein the at least one first reactor and the at least one connecting piece are in a coaxial arrangement and/or the second reactor and the inner tube are in a coaxial arrangement.

4. The apparatus of claim 1, wherein the first section and/or the second section each have at least two inlets.

5. The apparatus of claim 1, wherein the apparatus has at least two first reactors each having a connecting piece opening tangentially into the outer gas space of the second reactor.

6. The apparatus of claim 1, wherein the at least one connecting piece has a constant diameter, and the at least one first reactor and/or the second reactor each have a constant reactor diameter.

7. The apparatus of claim 1, wherein the inner tube has an open end and a closed end, and the open end is disposed at the outlet of the second reactor, or
wherein the inner tube has two open ends, and one of the two open ends forms the outlet of the second reactor.

8. The apparatus of claim 1, wherein the second reactor has a conical end and the outlet is disposed at the conical end.

9. The apparatus of claim 1, wherein the at least one connecting piece and/or the inner tube are equipped with a vibration apparatus.

10. The apparatus of claim 1, wherein at least parts of the first outer face of the at least one first reactor and/or the at least one connecting piece are equipped with a heating apparatus.

11. The apparatus of claim 1, wherein the at least one first reactor is made of steel, bronze, or aluminum, and/or the at least one connecting piece is made of graphite, quartz glass, tungsten, or molybdenum.

12. The apparatus of claim 1, wherein the inner tube extends from the second end face of the second reactor that is closer to the at least one connecting piece up to a length within a range from 50% to 70% of a total length of the second reactor.

13. A method of conducting a chemical reaction using the apparatus of claim 1, wherein the method comprises:
forming or converting a solid material by the chemical reaction,
wherein the chemical reaction is selected from the group consisting of pyrolysis of hydrocarbons, production of acetylene, reforming of hydrocarbons, pyrolysis of hydrogen sulfide, pyrolysis of ammonia, and hydrogasification of carbon.

14. The method as claimed in of claim 13, characterized in that further comprising:
tangentially feeding the first input gas into the at least one first reactor at the at least one inlet of the first section;
tangentially feeding the second input gas into the at least one first reactor at the at least one inlet of the second section;
mixing the first and second input gases in the at least one first reactor;
forming a plasma under the action of the electromagnetic waves in the at least one first reactor; and
withdrawing a product stream from the second reactor at the outlet,
wherein the first section is disposed on an opposite side of the window from the at least one connecting piece.

15. The method of claim 14, further comprising tangentially conducting the plasma into the second reactor.

16. The method of claim 13, wherein the product stream is at least partly recycled into the at least one first reactor.

17. The method of claim 13, further comprising transferring heat from the product stream to the first input gas and/or the second input gas.

18. The apparatus of claim 1, wherein the at least one connecting piece exits from the at least one first reactor at one of the first end faces and opens into the outer gas space of the second reactor at the second outer face in the tangential direction.

19. The apparatus of claim 1, wherein the window is made of quartz, alumina, boron nitride, or polytetrafluoroethylene.

20. The apparatus of claim 1, wherein the second reactor has the outlet at one of the second end faces for removal of the product stream.
